(19) United States
(12) Patent Application Publication
(10) Pub. No.: US 2017/0128642 A1
(43) Pub. Date: May 11, 2017

(54) NEGATIVE PRESSURE WOUND THERAPY DEVICE

(71) Applicant: KALYPTO MEDICAL, INC., Mendota Heights, MN (US)

(72) Inventor: John Buan, Maple Grove, MN (US)

(21) Appl. No.: 15/413,132

(22) Filed: Jan. 23, 2017

Related U.S. Application Data
(63) Continuation of application No. 13/760,610, filed on Feb. 6, 2013.
Application No. 13/760,610 is a continuation of application No. 12/455,013, filed on May 27, 2009, now abandoned.
(60) Provisional application No. 61/128,957, filed on May 27, 2008.

(51) Int. Cl. A61M 1/00; A61L 15/58; A61L 15/18; A61L 15/28; A61F 13/02; A61L 15/26

(52) U.S. Cl. CPC A61M 1/0088; A61F 13/0206; A61F 13/0213; A61F 13/0216; A61L 15/26; A61L 15/18; A61L 15/28; A61L 15/58; A61M 2205/15; A61M 2205/3331

(57) ABSTRACT

A wound therapy device which may include a gasket distally spaced from an absorptive pad and an edge of a backing material. The device may also include an adaptor, a tube portion with a connector located distally from a port hole in the backing material, and at least one viewing portal disposed over the absorptive material allowing the absorptive material to be seen from the second side of the backing material. Also, a method of determining a saturation level of such a device.

### RELATED APPLICATIONS

This application is a continuation of U.S. patent application Ser. No. 12/455,013 filed on May 27, 2009, which claims priority to U.S. Provisional Application No. 61/128,957, filed May 27, 2008, the entirety of both of which are incorporated herein. The invention in the present application relates, generally, in subject matter to the devices disclosed in Applicant's own U.S. Patent Pre-Grant Publication Nos. 2007/0265585 and 2007/0265586.

### TECHNICAL FIELD

The present invention relates, in general, to a device and method for wound therapy that is capable of treating a variety of chronic and acute wound types, including, but not limited to, infection wounds, venous ulcers, arterial ulcers, diabetic ulcers, burn wounds, post amputation wounds, surgical wounds, and the like. Specifically, the present disclosure is related to wound treatment devices and methods that utilize negative pressure therapy.

### BACKGROUND

Negative pressure therapy has been one method used for the treatment of a variety of wounds by practitioners in the art. Conventional negative pressure therapy devices are generally large in size and often require the use of complicated equipment such as suction pumps, vacuum pumps and complex electronic controllers.

Since the negative pressure therapy devices (e.g., dressings) utilize negative pressure, it is desirable to minimize the opportunity for leaks in same, so as to prevent increased damage to the patient and/or wound, or unnecessarily prolonged damage to the patient and/or wound.

Additionally, since negative pressure therapy devices (e.g., dressings) are usually wrapped with, for example, gauze, ace bandages, compression stockings, etc., it would be desirable and beneficial for the connection point between the pump and the negative pressure therapy device to be disposed distally from the negative pressure therapy device. This may increase the comfort of the patient, as well as allow for a limited amount of access to the negative pressure therapy device while it is wrapped.

### BRIEF SUMMARY OF THE INVENTION

In accordance with an embodiment of the invention, a wound therapy device includes a backing material, a port hole disposed in the backing material, an absorptive pad disposed on the first side of the backing material such that a portion of the absorptive pad is disposed under the port hole, and, a gasket disposed on the backing material distally between the absorptive pad and the edge.

The use and position of the gasket, along with other factors, are believed to decrease the chances for air pressure leaks. Thus, this will increase the effectiveness and usefulness of the negative pressure therapy device.

In another embodiment of the invention, the gasket is a hydrogel material.

In yet another embodiment of the invention, a wound therapy device also includes a wound interface layer, such as a silver plated mesh, disposed around an exposed portion of the absorptive pad.

In still another embodiment of the invention, a wound therapy device includes a backing material, a port hole disposed in the adhesive backing material, wherein an adaptor is disposed within the port hole and communicating with a connector with a tube having a length such that the connector is distally located from the port hole, an absorptive pad disposed on the first side of the backing material such that a portion of the absorptive pad is disposed under the port hole, and, a gasket disposed distally on the backing material between the absorptive pad and the edge.

By having the connector located distally from the port hole, the patient's comfort can be increased, and access to the connector will not require removal of bandages covering the negative pressure therapy device.

In another aspect of the invention, the invention is a method of making a wound therapy device which includes the steps of applying a port hole to an adhesive substrate, using the port hole as a registration point for applying a gasket material, applying a predetermined thickness of the gasket material in a predetermined shape at a distance around the port hole, applying a liner to the adhesive substrate, and, rolling the adhesive substrate.

In yet another aspect of the invention, the method of making a wound therapy device includes that the gasket material is applied by being poured.

### BRIEF DESCRIPTION OF THE DRAWINGS

The present invention will become more fully apparent from the following description and appended claims, taken in conjunction with the accompanying drawings. Understanding that the accompanying drawings depict only typical embodiments, and are, therefore, not to be considered to be limiting of the scope of the present disclosure, the embodiments will be described and explained with specificity and detail in reference to the accompanying drawings as provided below.

FIG. 1 is a cutaway side view of an embodiment of a wound healing device according to the present invention.

FIG. 2 is a top view of an embodiment of a wound healing device according to the present invention.

FIG. 3 is an exploded view of an embodiment of a wound healing device according to the present invention.

FIG. 4 is a back view of an embodiment of a wound healing device according to the present invention.

### DETAILED DESCRIPTION OF THE DRAWINGS

It will be readily understood that the components of the embodiments as generally described and illustrated in the Figures herein could be arranged and designed in a wide variety of different configurations. Thus, the following more detailed description of various embodiments, as represented in the Figures, is not intended to limit the scope of the present disclosure, but is merely representative of various embodiments. While the various aspects of the embodiments are presented in drawings, the drawings are not necessarily drawn to scale unless specifically indicated.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

In the following description, numerous specific details are provided to provide a thorough understanding of embodiments of the invention. One skilled in the relevant art will recognize, however, that the invention can be practiced without one or more of the specific details, or with other methods, components, materials, and so forth. In other instances, well-known structures, materials, or operations such as vacuum sources are not shown or described in detail to avoid obscuring aspects of the invention.

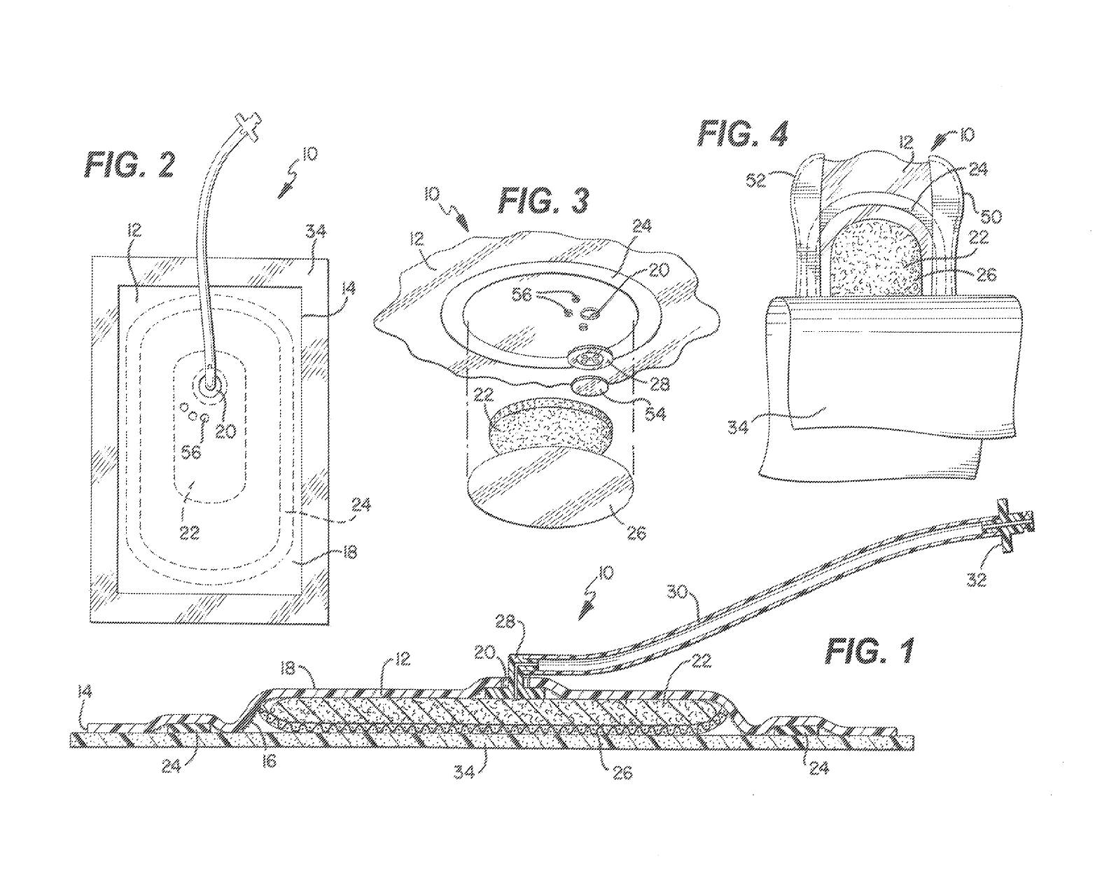

Referring now to FIGS. 1-4, a wound therapy device 10 is shown. A wound therapy device 10 includes a backing material 12 having a shape with an edge 14 and a first side 16 having an adhesive and a second side 18. The present invention contemplates multiple shapes including, but not limited to, circles, ovals, squares, and oblongs. The backing material 12 may be flexible to allow the device 10 to be contoured to the appropriate location of a wound. In addition, it is preferred that the backing material 12 be semi-permeable. What is meant by the term semi-permeable is that the backing material has breathability aspects that do not impact the ability to hold negative pressure relative to appropriate therapeutic treatment, as would be understood by those of ordinary skill in the art.

It is contemplated that a liner 34 is removably attached to a portion of the first side 16 of the backing material 12. In a preferred embodiment, a second liner 50 and a third liner 52 each disposed on a portion of the edge 14. This is to facilitate quick deploy and use of the device 10. For example, the liner 34 can be removed. The clinician or patient placing the device 10 can utilize second liner 50 and third liner 52 while placing the device 10 without touching the adhesive on the first side 16.

A port hole 20 is disposed in the backing material 18. The port hole 20 can have any shape and size. In an embodiment of the present invention, an adaptor 28 is disposed within the port hole 20. In a preferred embodiment of the invention, a fluid impermeable membrane 54 is disposed on the adaptor 28. The fluid impermeable membrane 54 prohibits fluids and other exudates from flowing from the device 10 to the source of the negative pressure. In a preferred embodiment, the fluid impermeable membrane 54 is GORE-TEX®; however, other materials are contemplated to be used.

A tube segment 30 allows for communication between the adaptor 28 and a connector 32. The connector 32 is connected (either directly or indirectly) to the source of the negative pressure (not shown). The tube segment 30 is long enough that the connector 32 is distally spaced from the adaptor 28 and/or port hole 20. Non-kinking tube material may be used. It is contemplated that the adaptor 28, tube segment 30 and connector 32 is comprised of one structure or multiple structures connected. By moving the connector 32 away from the port hole 20, it is believed that the device will increase the comfort of the patient, since the connection between the device and pump does not have to be located under gauze or other wrapping material such as Unna Boot or COBAN® which typically wraps around the device. Additionally, if the connection between the pump and device needs to be broken, the wrapping material does not need to be removed.

Returning to FIGS. 1-4, an absorptive pad 22 is disposed adjacent the first side 16 of the backing material 12. The absorptive pad 22 is capable of absorbing exudates and liquid from a wound, while continuing to allow the device to communicate negative pressure to the wound. The absorptive pad 22 may be a material such as sponges, foams, fibers, wicking fibers, hollow fibers, beads, fabrics, or gauzes, super-absorbent materials including super-absorbent polymers in various forms, absorbent foams, gelling agents such as sodium carboxy methyl cellulose, packing materials, and/or combinations thereof. Since the absorptive pad 22 allows the communication of the negative pressure from a pump to the wound, the absorptive pad 22 is disposed under the port hole 20. By used of the term “under,” it is meant that when the device 10 is viewed from a top view (such as in FIG. 2), a portion of the absorptive pad 22 is disposed beneath the port hole 20. In addition, it is contemplated that the absorptive pad 22 need not be directly beneath or under the port hole 20, i.e., other structures may be disposed between the two structures.

In addition to the port hole 20, it is contemplated that the device 10 includes at least one viewing portal 56 in the backing material 12. Since the absorptive pad 22 retains the exudates and fluids removed from the wound, the viewing portal 56 allows a clinician or patient to determine if the absorptive pad 22 is saturated or not. It is preferred that the viewing portal 56 be disposed between the wound and the port hole 20. In addition, since certain backing materials are non-transparent it is contemplated that a semi-transparent material be disposed over the viewing portal 56 so as to prevent any exudates from leaking out.

It is contemplated that the device 10 also includes wound interface layer 26, or other similar structure, disposed around a portion of the absorptive pad 22. The wound interface layer 26 will allow epithelialization and reduce wound tissue adherence to the device. In one embodiment, the wound interface layer 26 may comprise a silver plated mesh, such as one that is currently commonly available and known as SILVERION®.

A gasket 24 is disposed on the backing material 12, and more particularly on the first side 16 of the backing material 12 with the adhesive. The gasket 24 is disposed distally between the edge 14 of the backing material 12 and the absorptive pad 22. It is contemplated that the gasket 24 be disposed immediately adjacent to the absorptive pad 22.

The gasket 24 has a thickness, and it is contemplated that the thickness of the gasket 24 is between 3 to 5 mils and the width of the gasket 24 is approximately ⅜ of an inch.

In one embodiment of the invention the gasket 24 is a hydrogel. Such materials are currently available from Katecho, in Des Moines, Iowa (USA). It is preferred that the gasket 24 be a material that be biocompatible with skin. In addition the gasket 24 material should mildly adhere to the skin, but not adhere to the skin in the same manner as the adhesive on the backing material. In addition, the gasket 24 material should be mildly flowable. Furthermore, the gasket 24 material should be non-reactive to normal medical device sterility processes. Another contemplated material is a silicone gel; however, it is currently believed to be too cost prohibitive to utilize the silicone gel.

It is preferred that the gasket 24 and the absorptive pad 22 have the same (relatively) shape. It is most preferred that the shape is an oval. Moreover, it is important that the gasket 24 be sized such that the wound is entirely disposed within the gasket 24.

Such a gasket 24 is believed to minimize the possibility of air pressure leaks, and thus increase the efficiency of the device.

In addition to the devices described here, an embodiment of the invention is a method of making a wound therapy device. The method includes the steps of:
applying a port hole to adhesive substrate;
using the port hole as a registration point for applying a gasket material;
applying a predetermined thickness of the gasket material in a predetermined shape at a distance around the port hole;
applying a liner to the adhesive substrate;
rolling the adhesive substrate.

The step of applying a port hole to an adhesive substrate may include punching, cutting, slicing, removing or any other action that results in a port hole being made in an adhesive substrate. The adhesive substrate may be the backing material described above.

By using the port hole as a registration point, it is meant that the port hole location is used as the position to determine where the gasket material is to be applied. Once it is determined where the gasket material is to be applied, the gasket material can be applied at a distance in a predetermined shape around the port hole. It is contemplated that the gasket material is applied by being poured. Additionally, the gasket can be applied in any number of predetermined shapes and thickness.

A liner can be applied to the adhesive substrate and the combination of the adhesive substrate and liner can be rolled and subsequently stored. The resulting combination of adhesive substrate and liner material can then be cut into smaller individual wound therapy devices, and further steps such as providing an absorptive pad and/or providing an adaptor can be accomplished.

It is also contemplated that the method includes the steps of providing the second liner and third liner each on a portion of an edge of the adhesive substrate, such as described above.

Since after use the wound therapy devices usually contain bodily waste typically comprising exudates and liquid from a wound, they are disposable, and thus, decreasing the costs of manufacturing, would be beneficial and is believed to be desirable. Additionally increasing the manufacturability may also be desirable.

Currently, the devices that are described herein are manufactured with a hybrid method, involving some steps that are performed by machine, and other steps that are performed by hand. Such a process may include taking stock material for the gasket and, after cutting the stock material, placing the gaskets by hand onto the backing material. However, it is contemplated that the entire method of manufacture be performed by machines, by hand or a hybrid thereof.

Without further elaboration, it is believed that one skilled in the art can use the preceding description to utilize the present disclosure to its fullest extent. The examples and embodiments disclosed herein are to be construed as merely illustrative and not a limitation of the scope of the present disclosure in any way. It will be apparent to those having skill in the art that changes may be made to the details of the above-described embodiments without departing from the underlying principles of the disclosure provided herein. In other words, various modifications and improvements of the embodiments specifically disclosed in the description above are within the scope of the appended claims. Note that elements recited in means-plus-function format are intended to be construed in accordance with 35 U.S.C. §112 ¶6. The scope of the invention is therefore defined by the following claims.

1.-33. (canceled)

34. A wound therapy device comprising:
a backing material having a shape with an edge and a first side having an adhesive and a second side, the backing material configured to maintain negative pressure under the first side of the backing material;
a port hole disposed in the backing material and an adaptor disposed over the port hole configured to communicate negative pressure to the first side of the backing material from a source of negative pressure;
an absorptive pad disposed under the first side of the backing material such that a portion of the absorptive pad is disposed under the adaptor;
a wound interface layer disposed under the absorptive pad;
a fluid impermeable material disposed above the absorptive material and covering the port hole, the fluid impermeable material configured to stop exudate from flowing to the source of negative pressure; and
a gasket disposed on the first side of the backing material between the absorptive pad and the edge of the backing material.

35. The device of claim 34, wherein the gasket comprises a hydrogel.

36. The device of claim 34, wherein the gasket comprises a silicone gel.

37. The device of claim 34, wherein the absorptive pad has a shape and the gasket has a shape, and wherein the shape of the absorptive pad is substantially the same as the shape of the gasket.

38. The device of claim 34, wherein the gasket is disposed immediately adjacent the absorptive pad.

39. The device of claim 34, wherein the wound interface layer is a silver plated mesh.

40. The device of claim 34, wherein the backing material is semi-permeable.

41. The device of claim 34, further comprising a tubing portion having a first end communicating with the adaptor and second end communicating with a connector.

42. The device of claim 34, wherein the adaptor further comprises a fluid impermeable membrane.

43. The device of claim 34, wherein the absorptive pad comprises a gelling agent.

44. The device of claim 43, wherein the gelling agent comprises a carboxy methyl cellulose material.

45. The device of claim 43, wherein the absorptive pad further comprises foam.

46. The device of claim 34, wherein the shape of the absorptive pad is square.

47. A wound therapy device comprising:
a backing material having a shape with an edge and a first side having an adhesive and a second side, the backing material configured to maintain negative pressure under the first side of the backing material, wherein the shape of the edge is oval;
a port hole disposed in the backing material and an adaptor disposed in the port hole configured to communicate negative pressure to the first side of the backing material from a source of negative pressure;
a gauze pad disposed under the first side of the backing material such that a portion of the gauze pad is disposed under the adaptor;
a wound interface layer disposed under the gauze pad, wherein the wound interface layer comprises a mesh; and
a gasket disposed on the first side of the backing material between the gauze pad and the edge of the backing material.

48. The device of claim 47, wherein the gasket comprises a hydrogel.

49. The device of claim 47, wherein the gauze pad has a shape and the gasket has a shape, and wherein the shape of the gauze pad is substantially the same as the shape of the gasket.

50. The device of claim 47, wherein the shape of the gauze pad is oval.

51. The device of claim 47, wherein the gasket has a width of approximately ⅜ of an inch.

52. The device of claim 47, wherein the gasket has a thickness of approximately 30 mils.

53. The device of claim 47, wherein the gasket is disposed immediately adjacent the gauze pad.

54. The device of claim 47, wherein the wound interface layer is a silver plated mesh.

55. The device of claim 47, wherein the backing material is semi-permeable.

56. The device of claim 47, further comprising a tubing portion having a first end communicating with the adaptor and second end communicating with a connector.

57. The device of claim 47, wherein the gasket comprises a silicone gel.

58. The device of claim 47, wherein the backing material is non-transparent.

59. A method of treating a wound with reduced pressure, the method comprising:
applying a dressing over the wound to form a sealed space over the wound, the dressing comprising:
a backing material having a shape with an edge and a first side having an adhesive and a second side, the backing material configured to maintain negative pressure under the first side of the backing material, wherein the shape of the edge is oval;
a port hole disposed in the backing material and an adaptor disposed in the port hole;
a gauze pad disposed under the first side of the backing material such that a portion of the gauze pad is disposed under the adaptor;
a wound interface layer disposed under the gauze pad, wherein the wound interface layer comprises a mesh, and
a gasket disposed on the first side of the backing material between the gauze pad and the edge of the backing material;
wherein both the adhesive on the first side of the backing material and the gasket adhere the dressing to skin surrounding the wound; and
supplying negative pressure through the adaptor to the first side of the backing material from a source of negative pressure.
